(19) United States
(12) Patent Application Publication
(10) Pub. No.: US 2004/0234508 A1
(43) Pub. Date: Nov. 25, 2004

(54) Arrangement with artificial neurons for describing the transmission behavior of a nerve cell to be excited and method for determining the transmission behavior of a nerve cell to be excited using artificial neurons

(76) Inventors: Bernd Schurmann, Haimhausen (DE); Martin Stetter, Munchen (DE); Gustavo Deco, Neubiberg (DE); Jan Storck, Germering (DE); Silvia Corchs, Munchen (DE)

Correspondence Address: SCHIFF HARDIN, LLP, PATENT DEPARTMENT, 6600 SEARS TOWER, CHICAGO, IL, 60606-6473, US

(21) Appl. No.: 10/489,535

(22) PCT Filed: Sep. 11, 2002
(86) PCT No.: PCT/DE02/03381

(30) Foreign Application Priority Data
Sep. 12, 2001 (DE) 101 44 920. 8

(51) Int. Cl. G06E 1/00; G06E 3/00; G06N 3/02; C12N 5/08

(52) U.S. Cl. 424/93.7; 435/368; 706/15

(57) ABSTRACT

In an arrangement and a method for the determination and description of the transmission behavior of a nerve cell, using artificial neurons, a first artificial neuron describes an exciter nerve cell and has a first input to which a first input signal is supplied representing external synaptic activity, a second input to which a second input signal is supplied representing internal synaptic activity, and an output at which an output signal, representing action potential activity, is emitted. A second artificial neuron generates the second input signal corresponding to internal synaptic activity.

### BACKGROUND OF THE INVENTION

1. Field of the Invention

The present invention relates to the determination and description of the transmission behavior of an exciter nerve cell using artificial neurons.

2. Description of the Prior Art

Zell, A., “Simulation neuronaler Netze” [Simulation of neural networks], pp. 35 to 51, pp. 55 to 86, Addison-Wesley Longman Verlag GmbH, 1994, 3rd unrevised reprint, R. Oldenbourg Verlag, ISBN 3-486-24350-0, 2000 describes how nerve cells (neurons) and their biological functionality, i.e. their biological behavior, can be simulated by means of artificial neurons.

FIG. 2 schematically illustrates the structure of such a nerve cell 200 which can be simulated by an artificial neuron.

This nerve cell 200, a basic building block of the brain, has dendrites 210, i.e. thin, tubular and usually heavily branched projections with which the nerve cell 200 picks up input signals. These input signals, called synaptic activity, are processed in the nerve cell 200, in particular in its cell body 220, and passed on as electrical nerve impulses or electrical potentials, also known as action potential or “spiking” activity.

The electrical nerve impulses are passed on via a nerve fiber 230, called an axon, which is branched at its end 231.

At the end of these branchings 231 it forms thickenings 240, the synapses 240, via which the nerve cell 200 distributes its electrical nerve impulses to downstream nerve cells linked to it.

Clearly a synapse 240 is therefore a contact point between two nerve cells or the contact point between an end of an axon 230 of one neuron and a dendrite 210 of another neuron.

The Zell article also describes the distinction between two types of synapses or nerve cells, an exciter synapse 251 or nerve cell and an inhibitory synapse 252 or nerve cell.

Inhibitory synapses 252 reduce electrical potentials to be transmitted or passed on, exciter synapses 251 increase electrical potentials to be transmitted.

Further descriptions of the structure and functionality of a nerve cell as well as nerve conduction are given in the Zell article.

Also known from the Zell article is an artificial nerve cell (artificial neuron) which simulates a (biological) nerve cell.

Clearly an artificial neuron of this kind is a mathematical mapping which maps an input quantity of the artificial neuron to an output quantity of the artificial neuron in accordance with the transmission behavior of the biological nerve cell.

Based on the biological model, an artificial neuron comprises three components: the cell body, the dendrites which accumulate input signals to the artificial neuron, and the axon which passes on the output signal of the artificial neuron to the outside, is branched and makes contact with the dendrites of downstream artificial neurons via synapses.

Synapse strength or synapse type generally is represented by a numerical value or its sign. This value is termed the connection weight.

A transmission behavior or the mapping behavior of an artificial neuron can be mapped according to the biological model as described in the Zell text.

Compared to the biological model, the simulation of a nerve cell by an artificial neuron of this kind is greatly idealized and therefore inexact, i.e. the transmission behavior that can be implemented by the artificial neuron does not conform or conforms imprecisely to that exhibited by the nerve cell.

Thus in the Zell text, for example, interaction effects between nerve cells which influence the transmission behavior of a nerve cell, particularly between an exciter and an inhibitory nerve cell, are not taken into account for idealization by an artificial neuron.

Further remarks concerning artificial neurons and their functionality are given in the Zell text.

In addition, a further part of the Zell text (pgs. 87-114) describes how individual neurons can be linked together. Such an arrangement of interlinked neurons is known as a neural network. The basic principles of neural networks, e.g. various types of neural networks, training methods for neural networks and references to biological nerve cell arrangements, are described in this further part of the Zell text.

J. J. Binney, N. J. Dowrick, A. J. Fisher, M. E. J. Newman, “The Theory of Critical Phenomena”, Chapt. 6: Mean-Field Theory, Clarendon Press Oxford 1992 demonstrates how a mean field model can be used to describe a complex system. In a mean field model, stochastic interaction effects between components of a system are approximated by an average interaction effect. This enables stochastic systems that cannot be described analytically to be reduced to describable deterministic systems.

C. Koch, I. Segev (eds.), “Methods of Neural Modeling: From Ions to Networks”, Chapt. 13: D. Hansel and H. Sompolinsky: “Modeling Feature Selectivity in Local Circuits”, MIT Press, Cambridge, 1998 shows how the mean field model can be used to describe a neuron structure.

A. W. Toga and J. C. Maziotta (eds.), “Brain Mapping: The Methods”, Chapt. 9: M. S. Cohen: “Rapid MRI and Functional Applications”, Academic Press 1996 describes the basic principles of functional magnetic resonance imaging or fMRI technology which is a further development of known magnetic resonance imaging.

Magnetic resonance (MR) imaging is a known imaging method which produces sectional images of the human body without subjecting it to X-rays, utilizing instead the behavior of body tissue in a powerful magnetic field. This enables pathological changes, e.g. in the brain or spinal cord, to be detected.

However, functional abnormalities in the body tissue, particularly in the patient's brain, cannot be detected using conventional magnetic resonance imaging.

This can be done by functional magnetic resonance imaging or fMRI technology.

fMRI enables neural activity in areas of a patient's brain to be measured indirectly. It measures the so-called BOLD (Blood Oxygenation Level Dependent) signal in individual areas of the brain, this signal bearing a relationship to the neural activity in the areas in question.

The result of such fMRI measurements shows the pattern of activity of the individual areas over a certain period of time, e.g. during cognitive sequences as the result of certain perceptual processes or motor tasks. Functional abnormalities in the brain therefore are implicitly contained in the fMRI signals measured.

The fMRI measurements are analyzed using mathematical methods to enable conclusions to be drawn directly about functional abnormalities in a brain and their causes.

These analysis methods are based mainly on mathematical models of the brain, the neuron structures in the brain and their (transmission) behavior.

A disadvantage of most of the analysis methods currently known and used is that they are insufficiently accurate and therefore result in incorrect conclusions for a diagnosis and/or are inefficient, i.e. the relevant analysis method is too slow and expensive to apply.

This disadvantage that applies to most of the known methods generally is attributable to the fact that the underlying modeling of the neuron structures in the brain, particularly the modeling of a neuron and its behavior, conforms only insufficiently to the biological model or reality.

Description for “fmri.pro” software for quantitative fMRI analysis, available on 7 Sep. 2001 from http://www.med.uni-muenchen.de/radin/html/arbeitsgruppen/fmri/ccfmri.html describes a software tool “fmri.pro” for an fMRI analysis method. Description of fMRI equipment, available on 7 Sep. 2001 from http://www.unipublic.unizh.ch/campus/uni-news/2001/0147/fmri.html describes a device for performing fMRI.

### SUMMARY OF THE INVENTION

An object of the invention is to provide an arrangement and an associated method with which the functionality, in particular the transmission behavior, of a nerve cell can be better simulated, i.e. with higher accuracy and conformity to the biological model, than is the case with simulation by the known artificial neuron.

This object is achieved in accordance with the invention by an arrangement with artificial neurons for describing a transmission behavior of an exciter nerve cell, by means of which transmission behavior an external and an internal synaptic activity is translated into an action potential activity, having:

a first artificial neuron describing the exciter nerve cell and having a first input for feeding in a first input signal corresponding to the external synaptic activity, having a second input for feeding in a second input signal corresponding to the internal synaptic activity, and having an output for feeding out an output signal corresponding to the action potential activity, and

a second artificial neuron connected to the first artificial neuron via a second input and describing an inhibitory nerve cell, said second artificial neuron producing the second input signal of the first artificial neuron.

The above object also is achieved in accordance with the invention by a method for determining a transmission behavior of an exciter nerve cell, by means of which transmission behavior an external and an internal synaptic activity is converted into an action potential activity, the following steps are performed using artificial neurons, whereon:

a first input of a first artificial neuron describing the exciter nerve cell is fed a first input signal corresponding to the external synaptic activity,

a second input of the first artificial neuron is fed a second input signal corresponding to the internal synaptic activity and produced by a second artificial neuron describing an inhibitory nerve cell and connected to the first artificial neuron via the second input, and

an output signal corresponding to the action potential activity is fed out at an output of the first artificial neuron,

with the transmission behavior being determined using the first and second input signal and the output signal.

In the invention, an artificial neuron is taken to mean a computing element which maps an input quantity, which is fed to the computing element using a specifiable mapping, to an output quantity that can be fed out by the computing element.

The mapping can be both a linear and a non-linear mapping and describes a function or a transmission behavior of an inhibitory or an exciter nerve cell.

In the invention, the exciter and the inhibitory nerve cell or the first and second artificial neuron can be regarded as an entity. Such an entity can be termed a “voxel”.

The particular advantage of the invention is that complex nerve cell structures of the kind present in the brain as well as their complex activity patterns can be described in a particularly simple and precise manner. The invention therefore allows reliable conclusions to be drawn regarding functional abnormalities of nerve cells or nerve cell structures.

The invention and the further developments described below can be implemented both in software and hardware, e.g. using a special electrical circuit.

It is additionally possible for the invention or a further development described below to be implemented by a computer-readable storage medium on which is stored a computer program which executes the invention or the further development.

The invention or any further development described below also can be implemented by a computer program product having a storage medium on which is stored a computer program which executes the invention or the further development.

In a further development of the invention, the first input signal is produced by another artificial neuron describing another exciter nerve cell and connected to the first artificial neuron via the first input.

In another structural further development, the first artificial neuron has a self-feedback loop for self-excitation by a self-excitation signal, by means of which self-feedback loop another output of the first artificial neuron is connected to a third input of the first artificial neuron.

For describing large-dimension nerve cell structures comprising an enormous number of individual nerve cells, as in the brain in particular, it is advisable for the exciter nerve cell described by the first artificial neuron to represent a plurality of exciter nerve cells, in particular a plurality of exciter nerve cells of the brain or in an area of the brain.

Accordingly it is advisable for the inhibitory nerve cell described by the second artificial neuron to represent a number of inhibitory nerve cells, in particular a plurality of inhibitory nerve cells of the brain or in an area of the brain.

In such a case it is possible for the number of exciter nerve cells and the number of inhibitory nerve cells to be assigned to the same specified area of the brain or to belong to the same area of the brain.

In general, nerve cells combined in a representative and described by a representative in this way are termed a population.

Accordingly, an inhibitory population is created by combining a number of inhibitory nerve cells, and an exciter population is created by combining a number of exciter nerve cells. An entity composed of an exciter and an inhibitory population may also be termed a voxel.

It is additionally advisable for a mean field model to be used for combining nerve cells to form a population. This means that individual interaction effects between individual nerve cells are no longer taken into account, but these individual interactions are approximated by an averaged interaction.

However, such a mean field model also can be used even for describing an individual nerve cell or for describing an individual voxel.

For describing such large-dimension nerve cell structures, as in the brain, for example, at least two but in general more voxels can be additionally interconnected via signal lines.

One possibility for such a connection of two voxels is that the output signal of the first artificial neuron of the first voxel is fed as the first input signal to the first artificial neuron of the second voxel and as another input signal to the second artificial neuron of the second voxel.

Clearly in this case the exciter nerve cell of the first voxel is connected both to the exciter nerve cell and to the inhibitory nerve cell of the second voxel.

However, other connection configurations are possible.

To improve the description accuracy of the biological model, it is advisable to provide non-linear mappings for the description of the exciter and inhibitory nerve cell by the relevant artificial neuron.

Thus it is advisable to determine relevant input and output nerve signals by means of measurements on the biological model, i.e. on a real exciter or a real inhibitory nerve cell, e.g. by means of current-frequency relationship measurements, to extract from them the corresponding mapping and to use it for describing the relevant nerve cell.

This mapping also can be established using physiological and/or neuroanatomical pre-knowledge.

It is also advisable, because biologically valid, to provide a differential formulation, i.e. a time variant formulation, e.g. of the type:

$$T \frac{ⅆ}{ⅆ t} {m}_{a} = - {m}_{a} + {g}_{a} ( {h}_{a} ) , \\ {h}_{a} = {\sum }_{b} {w}_{ab} {m}_{b} \qquad ( 1 )$$

where:

| | |
|---|---|
| τ | designates a time constant |
| d/dt | a differentiation with respect to a time variable t |
| a, b | an index for a nerve cell or for a population |
| m | a “spiking” activity |
| h | a synaptic activity |
| g(.) | a mapping rule. |

To improve the accuracy of the description of the biological model it is also advisable to weight the input signals and/or the output signal of the artificial neurons using weights or weighting values.

By means of such weights, which can be established using physiological and/or neuroanatomical pre-knowledge, a nerve conduction process, in particular a synaptic transmission, can be simulated for the biological model, e.g. the transmission of a nerve signal from an axon of a nerve cell via a synapse to a dendrite of a downstream nerve cell.

In a further development of the invention, as part of an idealization for simulating the biological model, all the weights are formulated as identical and/or time invariant.

In a further development of the invention, the output signal corresponding to an action potential or a “spiking” activity in the biological model is further processed in such a way that a number and/or a statistic of action potentials is determined from the output signal.

This makes it possible to draw conclusions directly concerning a degree or an intensity of an activity of the nerve cell described by the further development, i.e. the neural activity of the nerve cell.

In a further development the invention is used in functional magnetic resonance imaging (fMRI). In this further development a patient's brain activity is determined for a specified activation state of his brain in such as way that the activation state is described by a Blood Oxygenation Level Dependent (BOLD) signal which is measured during fMRI, and the output signal representing the brain activity is determined for the BOLD signal as the first input signal using the invention.

### DESCRIPTION OF THE DRAWINGS

FIG. 1 schematically illustrates nerve conduction in a voxel according to an embodiment of the present invention.

FIG. 2 schematically illustrates the anatomical structure of a nerve cell.

FIG. 3 illustrates an apparatus for performing an fMRI scan in accordance with an embodiment of the invention.

FIG. 4 schematically illustrates nerve conduction in an artificial voxel according to a further embodiment of the invention.

FIG. 5 is a flowchart showing the basic steps in nerve conduction in a voxel according to the embodiment of the invention shown in FIG. 4.

FIG. 6 schematically illustrates nerve conduction in two interlinked voxels according to an embodiment of the invention.

FIG. 7 schematically illustrates the transmission behavior of a voxel according to a further embodiment of the invention, on the basis of an input signal and an output signal which describe the transmission behavior of the voxel.

### DESCRIPTION OF THE PREFERRED EMBODIMENTS

Embodiment: Functional Magnetic Resonance Imaging

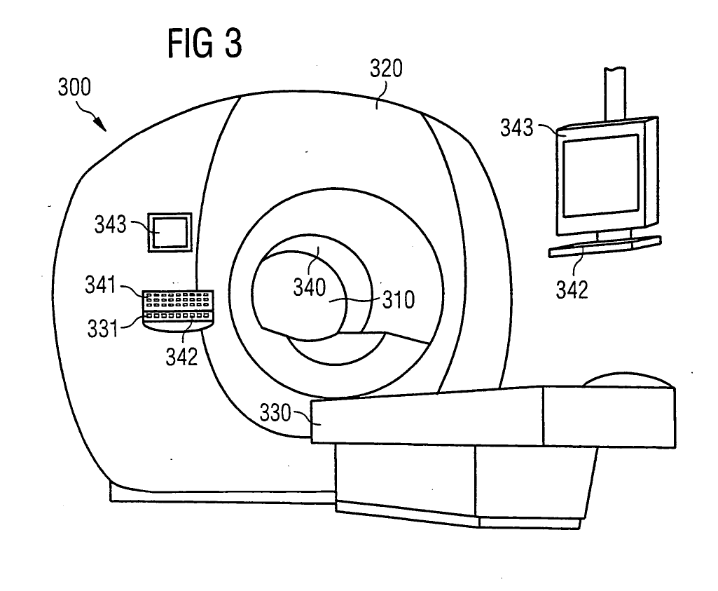

FIG. 3 shows a device 300 for performing functional scanning or magnetic resonance imaging (fMRI for short), a functional scanner or magnetic resonance tomograph 300.

The aforementioned Toga et al. text describes the basic principles of fMRI technology which is a further development of known magnetic resonance imaging.

The scanner 300 has a closed tube 310 incorporated in a magnet 320 in such a way that the latter produces a strong magnetic field in the tube 310.

The scanner 300 also has a patient table 330 which can be slid into the tube 310 and on which a patient is positioned for the scan.

The scanner 300 additionally has a control device 331, which enables the patient table 330 to be monitored and controlled during the scan, e.g. allowing the patient table 330 to be moved into the tube 320 in a controlled manner.

Other components of the scanner 300 are a measuring device 340 for measuring a BOLD (Blood Oxygenation Level Dependent) signal, an associated evaluation device 341 for analyzing the measured BOLD signal, in this case a high-performance computer, and an operating or interface device 342 for operating personnel as well as a display device 343 for displaying a scan result.

The components of the scanner 300 are functionally interconnected e.g. via signal or data lines over which data and signals can be transmitted.

Using the functional scanner 300 shown in FIG. 3, the neural activity in areas of a patient's brain can be measured on the basis of fMRI technology.

For this purpose the measuring device 340 is used to measure the BOLD (Blood Oxygenation Level Dependent) signal in individual areas of the patient's brain, this signal being related to the neural activity in the areas in question.

The result of such fMRI measurements shows the pattern of activity of the individual areas over a certain period of time, e.g. during cognitive sequences as the result of specific perceptual processes or motor tasks which the patient has to carry out during the scan. Function abnormalities in the patient's brain are therefore implicitly contained in the measured fMRI signals.

Using the evaluation device 341 which provides or carries out a corresponding analytical process, the fMRI measurements, i.e. the BOLD signals measured in individual areas of the brain, are analyzed to determine the brain activity in the form of corresponding activation patterns in the areas of the brain under examination, thereby allowing conclusions to be drawn directly regarding functional abnormalities in the brain and their causes.

The analytical process provided by the evaluation device 340 is based on a model of the brain, of the neuron structures in the brain and their (transmission) behavior, and this model is the basis in which the measured BOLD signal is analyzed and evaluated.

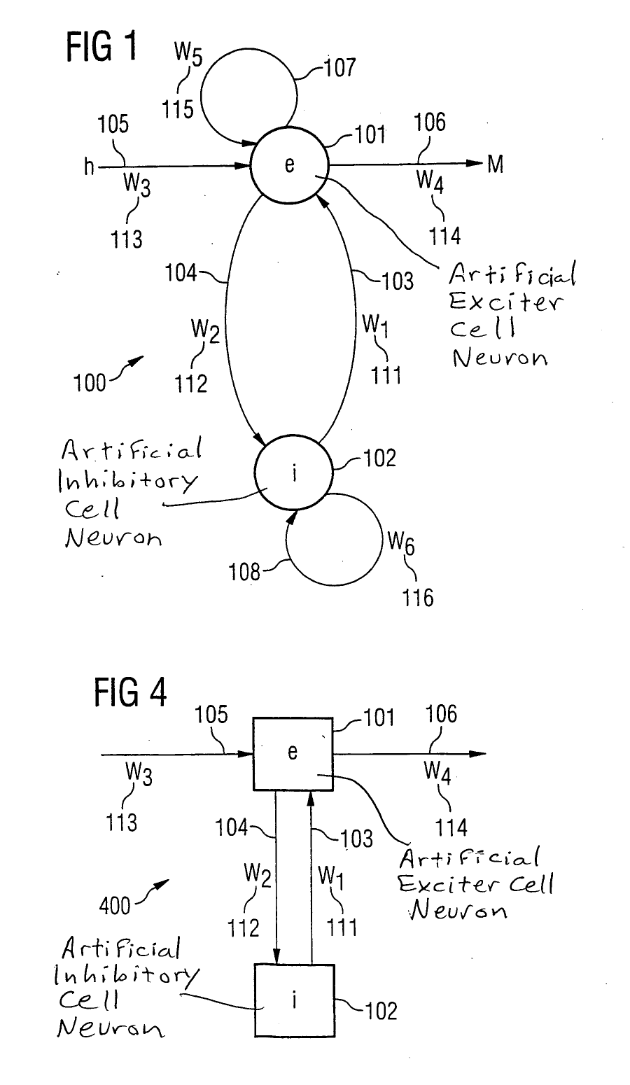

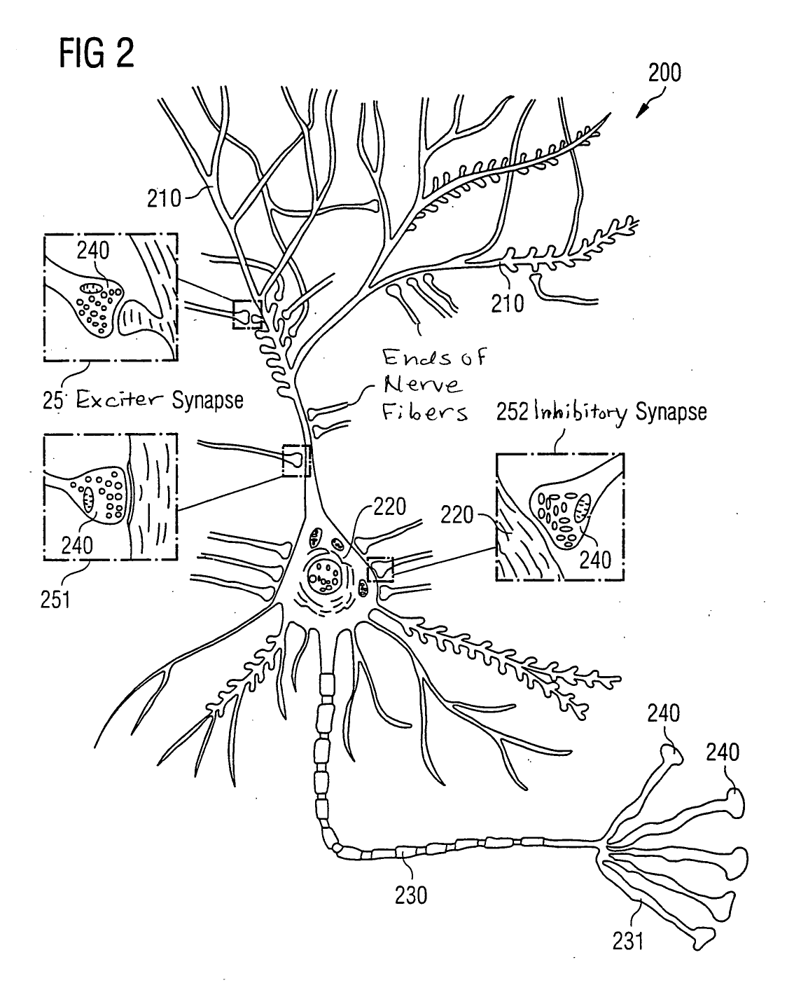

The principles of the analytical process and the model of the brain, of the neuron structures in the brain and their (transmission) behavior will now be explained with reference to FIGS. 1 and 4.

The results or the conclusions of a scan are displayed on the display device 343 and can be further processed by means of the operating and interaction device 342 in conjunction with the evaluation device 341.

Principles of the Analytical Process

In general in all of the figures a flow direction of a signal in a signal line, e.g. a flow direction of a nerve signal in a nerve line, is indicated by an arrow direction.

FIG. 1 shows a basic structure 100 of a complex nerve cell structure containing an enormous number of individual interconnected nerve cells in the brain.

In this basic structure 100, i.e. a voxel, an exciter nerve cell 101 is connected to an inhibitory nerve cell 102 via a first nerve line 103 and a second nerve line 104 in such a way that nerve signals of the inhibitory nerve cell 102 can be transmitted to the exciter nerve cell 101 and nerve signals of the exciter nerve cell 101 can be transmitted to the inhibitory nerve cell 101.

In addition, the exciter nerve cell 101 has a third nerve line 105 (dendrite) by means of which nerve signals of another nerve cell of another voxel can be transmitted to the exciter nerve cell 101 (post-synaptic activity h).

The exciter nerve cell 101 also has a fourth nerve line 106 (axon) by means of which nerve signals from the exciter nerve cell 101 can be transmitted to another second nerve cell of another second voxel (spiking activity M).

In addition, the exciter nerve cell 101 and the inhibitory nerve cell 102 have a self-exciter nerve line 107 and 108 respectively.

Each nerve line 103 to 108 is assigned a weight w (w1 to w6; 111 to 116) which represents synaptic transmission of the relevant nerve signal.

FIG. 4 shows the corresponding voxel model 400 of the voxel 100 shown in FIG. 1.

Essentially, structurally and functionally corresponding biological and associated modeled components are denoted by the same reference characters.

In this voxel model 400, a first artificial neuron 101 is connected to a second artificial neuron 102 via a first signal line 103 and a second signal line 104 in such a way that signals of the second artificial neuron 102 can be transmitted to the first artificial neuron 101 and signals of the first artificial neuron 101 can be transmitted to the second artificial neuron 101.

The first artificial neuron 101 additionally has a third signal line 105 by means of which signals of another artificial neuron of another voxel can be transmitted to the first artificial neuron 101 (post-synaptic activity h).

The first artificial neuron 101 also has a fourth signal line 106 by means of which signals from the first artificial neuron 101 can be transmitted to another second artificial neuron of another second voxel (spiking activity M).

Each signal line 103 to 106 is assigned a weight w (w1 to w4; 111 to 114) representing synaptic transmission of the nerve signal underlying the relevant signal.

FIG. 5 shows a listing 500 of the steps 501 to 504 occurring or carried out in the biological basic structure 100 and in the voxel model 400.

In a first step 501, a first input signal corresponding to the external synaptic activity is fed to a first input of a neuron describing the exciter nerve cell.

In a second step 502, a second input of the first neuron is fed a second input signal corresponding to the internal synaptic activity and produced by a second neuron describing an inhibitory nerve cell and connected to the first neuron via the second input.

In a third step 503, an output signal corresponding to the action potential activity is fed out at an output of the first neuron.

In a fourth step 504, a transmission behavior of the biological basic structure 100 or voxel or of the model 400 is determined using the first and second input signal and the output signal.

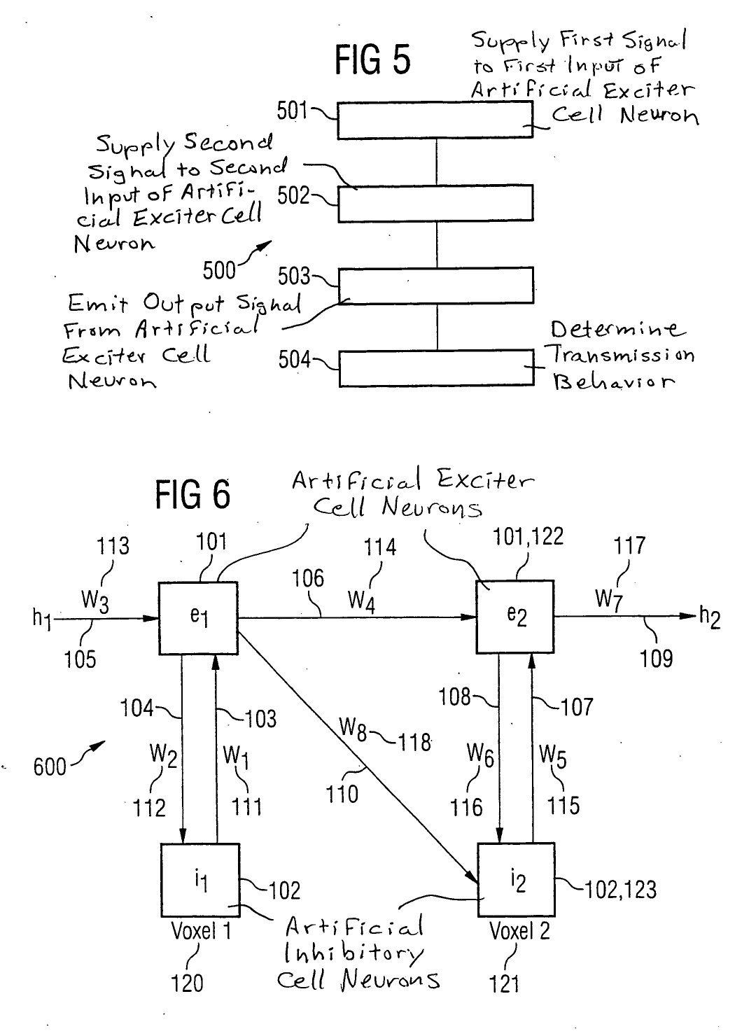

FIG. 6 shows a linking of two voxels or voxel models 120 and 121 in accordance with the voxel 100 or the voxel model 400.

Essentially, structurally and functionally corresponding biological and associated modeled components are denoted by the same reference characters in accordance with FIG. 1 and FIG. 4.

The two voxels or voxel models 120 and 121 are interlinked in such a way that the exciter nerve cell 101 of the first voxel 120 is connected both to the-exciter nerve cell 122 of the second voxel 121 and to the inhibitory nerve cell 123 of the second voxel 121 via lines 106 and 110.

Additional lines 107 to 109 and additional weights 115 to 118 are shown. For them the same applies as above.

The following functional relationships used as part of the analytical process for analyzing the BOLD signal can be specified for the voxels or voxel models described above (FIGS. 1, 4 and 6):

The functional relationships given below are based on the following non-limiting assumptions. These assumptions are not limiting but are merely used to simplify complex relationships. Fundamental aspects of the invention are unaffected by them.

Assumption 1: The nerve cell structures are based on relationships according to the mean field model as in the Binney et al. text and the Koch et al. text.

Assumption 2: All weights are assumed to be of equal size and time invariant. Weights of lines transmitting output signals of exciter neurons are positive. Weights of lines transmitting output signals of inhibitory neurons are negative.

The following designations are used:

| | |
|---|---|
| e | exciter neuron |
| i | inhibitory neuron |
| 1, 2 | index for voxel 1 or voxel 2 |
| a, b | index for a population 1 or population 2 |
| w | weight |
| h | synaptic activity, dynamic case |
| H | synaptic activity, static case |
| M or m | spiking activity (M: static; m: dynamic case) |
| t | time constant |
| d/dt | differentiation with respect to a time interval t |
| g(.) | mapping rule |

The following relations are valid:

$$T \frac{ⅆ}{ⅆ t} {m}_{a} = - {m}_{a} + {g}_{a} ( {h}_{a} ) , \\ {h}_{a} = {\sum }_{b} {w}_{ab} {m}_{b}$$

$${M}_{a} = g ( {\sum }_{b} {w}_{ab} {M}_{b} )$$

effective synaptic activity: Σ|h|

effective spiking activity: mₑ+mᵢ

Voxel 1

Mₑ₁=g(h₁−wMᵢ₁)

Mᵢ₁=g(wMₑ₁)

$${M}_{e1} = \frac{{h}_{1} + T ( w - 1 )}{1 + {w}^{2}}$$

$${M}_{i1} = w \frac{{h}_{1} + T ( w - 1 )}{1 + {w}^{2}} - T$$

static solution for voxel 1(T=0)

$${M}_{e1} = \frac{{h}_{1}}{1 + {w}^{2}}$$

$${M}_{i1} = \frac{{wh}_{1}}{1 + {w}^{2}}$$

Voxel 2:

Mₑ₂=g(h₂+wMₑ₁−wMᵢ₁)

Mᵢ₂=g(wMₑ₂+wMₑ₁)

W₂=h₂+2wMₑ₁+wMₑ₂+wMᵢ₂

Hₑ₂=h₂+wMₑ₁

Hᵢ₂=wMₑ₁

$${M}_{e2} = \frac{He - {wH}_{i}}{1 + {w}^{2}}$$

$${M}_{i2} = w \frac{{wH}_{e} + {H}_{i}}{1 + {w}^{2}}$$

static solution for voxel 2 (T=0):

$${M}_{e2} = \frac{1}{1 + {w}^{2}} ( {h}_{2} + \frac{w ( 1 - w )}{1 + {w}^{2}} {h}_{1} )$$

$${M}_{i2} = \frac{1}{1 + {w}^{2}} ( {h}_{2} + \frac{w + 1}{1 + {w}^{2}} {h}_{1} )$$

Analysis of a Measured BOLD Signal:

When scanning a selected area of the brain for functional abnormalities, the BOLD signal measured for that area is fed to the model described above as synaptic activity (He2+Hi2).

Using the model, the corresponding spiking activity Me2 is determined from this as a direct quantity for the neural activity in the selected area of the brain.

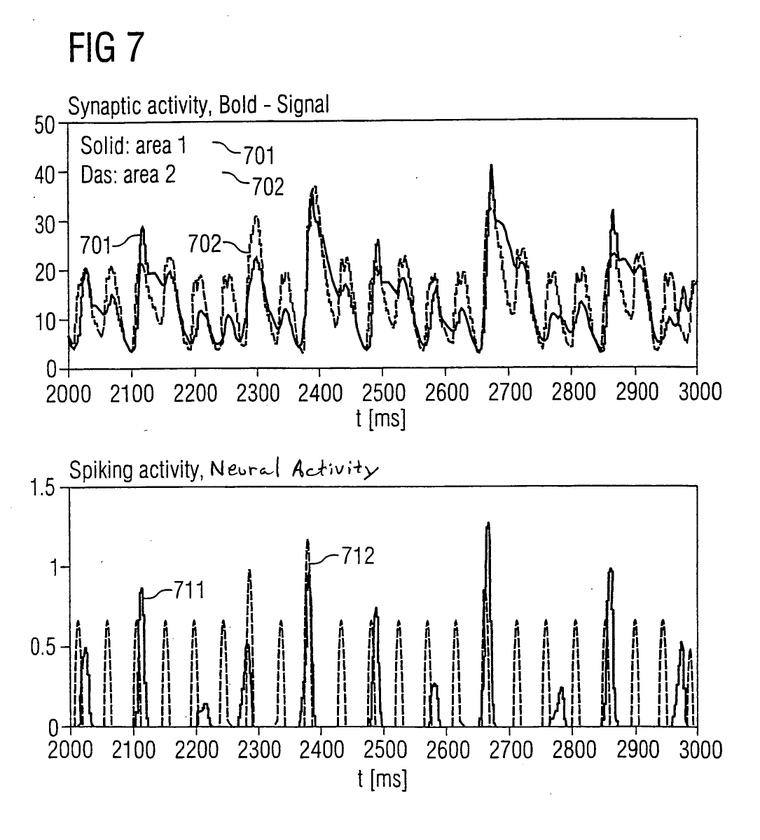

FIG. 7 shows as an example a first and second waveform 701 and 702 of such a BOLD signal for a first and a second area of the brain together with the first and the second associated waveform 711 and 712 of the first and second spiking activity or of the corresponding first and second neural activity pattern determined therefrom.

From the signal waveform of the spiking activity Me2 or from the activity pattern produced by the signal waveform in an area of the brain, functional abnormalities in that area can be detected.

Although modifications and changes may be suggested by those skilled in the art, it is the invention of the inventors to embody within the patent warranted heron all changes and modifications as reasonably and properly come within the scope of their contribution to the art.

We claim as our invention:

1-14. (cancelled)

15. An arrangement composed of artificial neurons for describing transmission behavior of an exciter nerve cell, said transmission behavior describing conversion of external and internal synaptic activity of the nerve cell into an action potential, said arrangement comprising:
a first artificial neuron describing the exciter nerve cell, having a first input for receiving a first input signal representing said external synaptic activity, a second input for receiving a second input signal representing said internal synaptic activity, and an output for emitting an output signal representing said action potential; and
a second artificial neuron connected to said second input of said first artificial neuron and describing an inhibitory nerve cell, said second artificial neuron generating said second input signal.

16. An arrangement as claimed in claim 15 comprising a further artificial neuron describing a further exciter nerve cell and connected to said first input of said first artificial neuron, said further artificial neuron generating said first input signal.

17. An arrangement as claimed in claim 15 wherein said first artificial neuron comprises a further output and a third input and a feedback loop connected between said further output and said third input for self-excitation of said first artificial neuron with a self-excitation signal generated at said further output.

18. An arrangement as claimed in claim 15 wherein said first artificial neuron describes an exciter nerve cell comprising a plurality of exciter nerve cells.

19. An arrangement as claimed in claim 18 wherein said first artificial neuron represents, as said plurality of exciter nerve cells, a plurality of exciter nerve cells assigned to a specified rain area.

20. An arrangement as claimed in claim 15 wherein said second artificial neuron describes an inhibitory nerve cell comprising a plurality of inhibitory nerve cells.

21. An arrangement as claimed in claim 20 wherein said second artificial neuron represents, as said plurality of inhibitory nerve cells, a plurality of inhibitory nerve cells assigned to a specified rain area.

22. An arrangement as claimed in claim 15 wherein said first artificial neuron weights at least one of said first input signal, said second input and said output signal according to a synaptic transmission.

23. An arrangement as claimed in claim 15 wherein said first artificial neuron weights at least two of said first input signal, said second input signal and said output signal with identical weights corresponding to a synaptic transmission.

24. An arrangement as claimed in claim 15 wherein said first artificial neuron weights at least one of said first input signal, said second input signal and said output signal with a time invariant weight corresponding to a synaptic transmission.

25. An arrangement as claimed in claim 15 comprising a processor supplied with said output signal for processing said output signal to determine a number of said action potentials.

26. An arrangement as claimed in claim 15 comprising a processor supplied with said output signal for determining a statistic associated with said action potentials.

27. An arrangement as claimed in claim 15 wherein said first artificial neuron employs a mean field model to represent said transmission behavior.

28. A method for determining transmission behavior of an exciter nerve cell, comprising the steps of:
generating a first input signal representing external synaptic activity;
supplying said first input signal to a first input of a first artificial neuron describing an exciter nerve cell;
generating a second input signal, representing internal synaptic activity, in a second artificial neuron describing an inhibitory nerve cell;
supplying said second input signal from said second artificial neuron to a second input of said first artificial neuron;
in said first artificial neuron, operating on said first and second input signals to generate an output signal dependent on transmission behavior of the exciter nerve cell described by said first artificial neuron.

29. A method as claimed in claim 28 wherein the step of generating said first input signal comprises obtaining a BOLD signal and using said bold signal as said first input signal.

30. A method as claimed in claim 29 comprising obtaining said bold signal from a patient by functional magnetic resonance imaging.
